(19) United States
(12) Patent Application Publication
(10) Pub. No.: US 2008/0283775 A1
(43) Pub. Date: Nov. 20, 2008

(54) Methods and Devices for Rapidly Forming Vitreous Ice-Jacketed Particle Droplets

(75) Inventors: Bruce Doak, Tempe, AZ (US); John Spence, Tempe, AZ (US); Uwe Weierstall, Phoenix, AZ (US)

Correspondence Address: MCDONNELL BOEHNEN HULBERT & BERGHOFF LLP, 300 S. WACKER DRIVE, 32ND FLOOR, CHICAGO, IL, 60606, US

(73) Assignee: The Arizona Board of Regents, a body corporate acting for and on behalf of Arizona State Universi, Tempe, AZ (US)

(21) Appl. No.: 11/908,319

(22) PCT Filed: Mar. 14, 2006
(86) PCT No.: PCT/US06/09045

§ 371 Date: Feb. 15, 2008

Related U.S. Application Data
(63) Continuation-in-part of application No. 11/358,237, filed on Feb. 21, 2006.
(60) Provisional application No. 60/662,273, filed on Mar. 15, 2005.
(60) Provisional application No. 60/662,467, filed on Mar. 15, 2005.
(60) Provisional application No. 60/662,273, filed on Mar. 15, 2005.
(60) Provisional application No. 60/662,467, filed on Mar. 15, 2005.

(51) Int. Cl. A61N 5/00; B01D 9/00; B01J 19/00; C12N 7/00

(52) U.S. Cl. 250/492.1; 23/293 R; 422/245.1; 435/235.1

(57) ABSTRACT

The present invention provides methods and devices for rapidly forming vitreous ice-jacketed particle droplets.

### CROSS REFERENCE

This application claims the benefit of U.S. provisional patent application Ser. Nos. 60/662,273 filed Mar. 15, 2005 and 60/662,467 also filed Mar. 15, 2005, and U.S. Utility patent application “Methods and devices for molecular structure determination” filed Feb. 21, 2006, Serial Number to be assigned, each of which is incorporated by reference herein in its entirety.

### STATEMENT OF GOVERNMENT INTEREST

This work was supported in part by National Science Foundation, Division of Biological Infrastructure, Program for Instrument and Instrument Development grant number 0429814, thus the U.S. government may have certain rights in the invention.

### FIELD OF INVENTION

This invention related generally to methods and devices for forming vitreous ice-jacketed particle droplets, and uses thereof.

### BACKGROUND OF THE INVENTION

Analysis and manipulation of particles, such as proteins or other biological molecules, often requires introducing or injecting the particle into vacuum, where the particle must maintain its native conformation. Examples of particle manipulation or analysis that may require particle injection into vacuum include molecular structure determination, spectroscopy, particle deposition onto a substrate (to produce, for example, sensor arrays), nanoscale free-form fabrication, formation of novel low temperature forms of particle-containing complexes, bombardment of particles by laser light, x-ray radiation, neutrons, or other energetic beams; controlling or promoting directed, free-space chemical reactions, possibly with nanoscale spatial resolution; and separating, analyzing, or purifying these particles.

Thus, methods and devices for promoting conformational stability of particles that are adapted for injection of the particle into vacuum would be of great benefit to these various fields.

### SUMMARY OF THE INVENTION

In a first aspect, the present invention provides method for rapidly forming a vitreous ice particle droplet beam, comprising contacting a hydrated particle droplet beam with a cryogenic gas in a first chamber at a temperature of 200K or lower, and at a pressure of 1 atm or lower, thereby forming a vitreous ice particle droplet beam. In a preferred embodiment, the method comprises passing a hydrated particle solution into the first chamber through a nozzle to form the hydrated particle droplet beam. In a further embodiment, the method further comprises injecting the vitreous ice particle droplet beam into a second chamber, wherein the second chamber has a temperature of 200K or lower, and wherein the second chamber has a pressure suitable to maintain the vitreous ice particle droplet beam. In a preferred embodiment, the second chamber comprises a vacuum chamber. In a further preferred embodiment, the cryogenic gas comprises helium.

In a further embodiment of this first aspect, the methods further comprise determining a structure of the particle by passing the vitreous ice particle droplet beam through a laser beam and a diffracting beam to produce a diffraction pattern for a first alignment of a plurality of particles in the vitreous ice particle droplet beam. In a preferred embodiment, particle structure determination further comprises (ii) passing the vitreous ice particle droplet beam through the laser beam and the diffracting beam to produce a diffraction pattern for a second alignment of a plurality of particles in the vitreous ice particle droplet beam; (iii) repeating step (ii) a desired number of times to produce diffraction patterns of further alignments of a plurality of the particles in the vitreous ice particle droplet beam; and (iv) determining a structure of the particle from a plurality of the diffraction patterns obtained from different alignments of the particle. In one embodiment, the diffracting beam comprises an electron beam; in another embodiment the diffracting beam comprises an X-ray beam.

In a preferred embodiment of all embodiments of the first aspect, the particle comprises a protein.

In a second aspect, the present invention provides a device for rapidly forming a vitreous ice particle droplet beam, comprising (a) a capillary tube comprising a distal end and a proximal end, wherein the distal end is in fluid connection with a reservoir adapted to contain a hydrated particle solution, and wherein the proximal end comprises a nozzle;

(b) a first chamber in fluid connection with the nozzle;

(c) a cryogenic gas source, in fluid connection with the first chamber;

(d) an injection tube comprising an inlet and an outlet, wherein the inlet is in fluid communication with the first chamber, and the outlet is adapted to be in fluid communication with a second chamber; and

(d) a housing.

In a further embodiment of this second aspect, the device further comprises a cooling system adapted to keep the first chamber at a temperature of 200K or lower. In a further embodiment, the device further comprises a second chamber, preferably a vacuum chamber, in fluid communication with the outlet of the injection tube.

In a third aspect, the present invention provides a device for carrying our serial diffraction, comprising:

(a) a vacuum chamber;

(b) a diffracting beam source in fluid communication with the vacuum chamber,

(c) a laser beam source in fluid communication with the vacuum chamber;

(d) a vitreous ice particle droplet beam source, comprising the device of the second aspect of the invention, wherein the outlet of the injection tube is in fluid communication with the vacuum chamber;

(e) a temperature control system for maintaining a temperature in the vacuum chamber of 200K or less; and

(f) a detector system in connection with the vacuum chamber;

wherein the diffracting beam source, the laser beam source, and the vitreous ice particle droplet beam source are positioned to permit beams directed from the diffracting beam source, the laser beam source, and the vitreous ice particle droplet beam source to intersect in the vacuum chamber in an intersecting volume of between 10 μl and 100 μL; and wherein the detector system is positioned so as to receive diffraction patterns from molecules in the molecule beam passing through the diffracting beam.

### BRIEF DESCRIPTION OF THE FIGURES

FIG. 1 is a diagram for an exemplary device for rapidly forming vitreous ice-jacketed particle droplets.

FIG. 2 is a diagram of an exemplary apparatus for performing serial diffraction. Parts list for FIG. 2:

1 Vacuum Chamber outline
2 Laser for molecular alignment (IR CW Fiber laser, 100 W, 1 micron wavelength)
3 Positioning device for alignment of laser beam, laser window and quarter wave plate polarizer
4 LN₂ cooled beam dump for droplet beam helps keep the vacuum<10⁻⁶ Torr
5 LN₂ feedthrough for cryoshield. The cryoshield prevents condensation from reaching the cooled CCD chip.
6 CCD camera
7 Vitreous ice particle droplet beam source
8 Laser beam for particle alignment
9 Ice-jacketed particle beam
10 Diffracting particle beam
11 In vacuum lens with positioning motors to focus alignment laser onto particle beam
12 Motors for particle beam alignment (allow movement of injection nozzle)
13 Observation port for visible alignment laser (a visible laser co aligned with the IR laser can be used to align the laser optics to the droplet beam)
14 Water-cooled infra-red laser beam dump
15 CCD camera readout and water-cooling

### DETAILED DESCRIPTION OF THE INVENTION

All publications, patents and patent applications cited herein are hereby expressly incorporated by reference for all purposes.

As used herein, the singular forms “a”, “an” and “the” include plural referents unless the context clearly dictates otherwise.

In a first aspect, the present invention provides methods for rapidly forming a vitreous ice particle droplet beam, comprising contacting a hydrated particle droplet beam with a cryogenic gas in a first chamber at a temperature of 200K or lower, and at a pressure of 1 atm or lower, thereby forming a vitreous ice particle droplet beam. Pressure ranges for use in the methods of the invention are dependent on various factors, including the size of the droplets, the cryogenic gas species used, and the distance the droplet travels through the gas. For example, for sub-micron diameter droplets in air, a preferred pressure is determined by the equation pL˜100 torr/cm, where “L” is the distance traveled, and where L=1 mm at p=1000 torr. In a preferred embodiment, pressures between about 0.01 atm and 1 atm are used.

Temperatures of 200K or lower can be used in these methods. Lower temperatures may be used, for example, in embodiments where particle alignment is desired (such as in structure determination embodiments described below). In such embodiments, smaller particles require lower temperatures. In an example where the particle comprises macromolecular species such as ribosome, a temperature of 200 K or lower is preferred. For smaller particles (e.g., Lysozyme), temperatures of ˜1 K or below are preferred, which can be obtained, for example, using liquid helium droplets in vacuum as described in more detail below.

The methods of this first aspect of the invention rapidly cool the solvated particles, thereby “quenching in” their native conformation, and produce a periodic, monodisperse, monodirectional stream of particles encased in a protective vitreous ice jacket that can be phase-locked to an external signal for further manipulation and/or analysis.

As used herein a “hydrated particle droplet beam” is a single file particle beam wherein the particles are suspended in a solvent. In a preferred embodiment, the solvent is water or standard buffer solutions. Other exemplary solvents that can be used with the invention include lipid solutions, such as those that allow the suspended particle to be brought into a particular state and conformation. In addition, the solvent can include, without limitation, buffering agents to set the pH and cryoprotectants to promote formation of vitreous ice and avoid crystalline ice as the droplets freeze. The preparation of particle beams is described in texts such as H. Pauly, Atomic Molecular Cluster Beams, Springer 2000, First Edition, Vol. 1, p. 149 section 4.1.1, incorporated herein by reference.

Any means for forming a single file (ie: monodirectional) hydrated particle beam can be used, such as by laminar flow of the hydrated particle solution through a nozzle. In a preferred embodiment, the method comprises passing a hydrated particle solution into the first chamber through a nozzle to form the hydrated particle droplet beam. As used herein, a “nozzle” is any mechanical device designed to control the characteristics of fluid flow as it exits from a source of hydrated particle solution into the first chamber. The nozzle is designed based on the specifics of the hydrated particle droplet beam desired, since the size of the droplet will be controlled by the nozzle aperture. In a preferred embodiment, the hydrated particle droplets range in diameter from between 5 μm and 5 nm, more preferably between 1 μm and 50 nm; even more preferably between 1 μn and 100 nm, and the diameter of interest is produced by use of a correspondingly sized nozzle aperture. It is further preferred that the nozzle be maintained at a temperature above the freezing temperature of the solvent.

In a further preferred embodiment, the methods of this first aspect further comprise applying a perturbation to the nozzle during passing of the hydrated particle solution into the first chamber, to facilitate breakup of the hydrated particle stream into individual droplets. Such a perturbation can be applied by any suitable means, including but not limited to a concentric piezoelectric element, allowing small oscillations to be applied to the nozzle tube, triggering the Rayleigh instability at a desired frequency and thereby locking the droplet production to a controllable phase. In a further embodiment, an intense, non-periodic excitation to produce droplets on demand is applied to the nozzle. For example, single intense pulses can be used to deliver “droplets on demand” as in ink-jet printers.

It is also preferred to control the charge on the ice-jacketed proteins produced by the hydrated molecule beam source, since a fluid flowing through a channel charges by electrokinetic effects even in the absence of an applied electric potential. Thus, in a preferred embodiment, the method further comprises placing an electric potential on the nozzle, to control charge of the particle droplets. It is further preferred that the electric potential comprises inductive charging controlled by a feed-back loop that measures current flow in the vitreous ice particle droplet beam.

As used throughout this application, the “particle” can be any biological or chemical compound, molecule, macromolecule, or larger assembly for which a method of rapid cooling to “quench-in” the native conformation is useful, including but not limited to proteins, nucleic acids, lipids, carbohydrates, chemical compounds, binding complexes (ex: protein-ligand complex), and viruses. In preferred embodiments, the particle comprises a protein or protein ligand-complex.

As used throughout this application, the “cryogenic gas” is any material (organic or inorganic) that remains a gas at 200K or below, including but not limited to helium, hydrogen, neon, argon, nitrogen, methane, ethane, and propane. The use of the co- or counter-flowing cryogenic gas in the first chamber serves to alter the temperature, size, and/or electrical charge state of the particle in the hydrated particle solution so as to form vitreous ice droplets.

The first chamber as recited herein can be any enclosed or partially enclosed body in which the hydrated particle droplet beam and the cryogenic gas can be contacted at a temperature of 200K or lower, and at a pressure of 1 atm or lower.

In a non-limiting example, a hydrated particle solution at near room temperature, possibly with cryoprotectant added to the solution to avoid crystallization, passes out of the nozzle and into the first chamber through which cold cryogenic gas flows in the opposite direction. In a preferred embodiment, 4 K helium at about 1 atm is used, but both higher temperatures and higher and lower pressures are also possible. The hydrated particle solution emerging from the nozzle breaks up due to Rayleigh instability and forms a stream of monodisperse, monodirectional droplets moving along the axis of the first chamber. By adjusting the concentration of particle molecules in the solution, the flow rate through the nozzle and the nozzle aperture, an appreciable fraction of the droplets can be made to contain one and only one particle molecule. For example, under optimum Rayleigh break-up from a 100 nm diameter nozzle to form a droplet beam traveling at 10 m/s: The volumetric flow rate is adjusted to 80 pL/s (picoliters per second) and a number concentration of 8×10⁻⁹ analyte molecules per water molecule is needed to have one analyte molecule in each droplet, on average. Those of skill in the art can determine other suitable conditions to generate droplets containing a single particle based on the teachings of the present invention.

As these droplets travel through the cold, dry cryogenic gas, they cool rapidly by transferring thermal energy to the counter-flowing cold cryogenic gas and by evaporation of water molecules from the droplets. The cooling rates will probably exceed 10⁸ K/s. The droplets freeze rapidly, forming vitreous or amorphous ice.

“Vitreous ice” refers to a particle in a glassy state, wherein the constituent atoms do not exhibit the long-range order that is characteristic of crystals, but do exhibit short-range order, and wherein the separation of atoms and/or the lengths of covalent bonds are very close to their typical equilibrium distances. The formation of vitreous rather than crystalline ice (due to the rapid cooling as taught herein) serves to “lock in” the native particle conformation, and thus to prevent denaturation.

The resulting “vitreous ice particle droplet beam” is also a single file particle beam, wherein the particles are protected by a vitreous ice jacket. The ice jacket may range in thickness from several micrometers down to less than one nanometer; preferably with a diameter of between 0.2 nm and 1 μm, and preferably between 1 nm and 1 μm. The particles are jacketed by vitreous ice and not subjected to high pressures or temperatures. The allowable pressures and temperatures depend critically on, for example, the individual molecule and the pH of the solution, but it appears that pressures above a few kbar and temperatures significantly above room temperature (but still well below the boiling point of water) will denature most proteins as taught by Hummer et al. [G. Hummer, et al., Proc. Natl. Acad. Sci. USA, 95, 1552-1555 (1998)] and by Heremans and Smeller [K. Heremans and L. Smeller, Biochimica et Biophysica Acta B1386B, 353-370 (1998)].

In a further embodiment, the methods of this first aspect of the invention further comprise injecting the vitreous ice particle droplet beam into a second chamber, wherein the second chamber has a temperature of 200K or lower, and wherein the second chamber has a pressure suitable to maintain the vitreous ice particle droplet beam; preferably the second chamber comprises a vacuum chamber and the pressure in the vacuum chamber is between about 10⁻⁵ and 10⁻⁸ torr. As is well known in the art, a vacuum chamber is a device to create a partial volume of space that is empty of matter and radiation, including air, so that gaseous pressure is much less than standard atmospheric pressure. Any such vacuum chamber can be used in conjunction with the methods of the invention, so long as a temperature of 200K or below in the vacuum chamber can be maintained.

In a preferred embodiment, the injecting comprises passing the vitreous ice particle droplet beam through a capillary with an inner diameter of 100 μM or less, and injecting the vitreous ice particle droplet beam from the capillary into the vacuum chamber. The primary purpose for using the capillary is to minimize cryogenic gas flow from the high pressure first chamber (ie: the “drying” section) into the vacuum chamber, and to promote free-jet expansion cooling. Helium is the most efficacious cryogenic gas for free-jet expansion of gases containing heterogeneous nucleation centers, which is precisely what the vitreous ice particle droplets in the beam will comprise. Droplets arrive at an inlet to the capillary tube traveling in essentially a straight line (actually a slightly parabolic curve due to gravitational acceleration). Given sufficient heat transfer within the first chamber, they reach the inlet with a temperature of about 4K. They pass through the capillary tube in this straight line, accompanied by a gas of helium atoms expanding from the ˜1 atm pressure of the first chamber into vacuum at the proximal end of the capillary. This pressure gradient accelerates the cryogenic gas and its pressure drops accordingly. At the proximal end of the capillary, the cryogenic gas undergoes a free-jet expansion into the surrounding vacuum under any remaining pressure difference. The vitreous ice droplets continue along their straight-line paths but the cryogenic gas atoms form a broad expansion fan radiating outward from the capillary exit. As the cryogenic gas expands, it cools and accelerates. There will always be “slip” between the droplet velocity and the cryogenic gas velocity, however, so that the cryogenic gas atoms overtake the droplets. If the temperature of the cryogenic gas falls low enough during the expansion, the cryogenic gas will condense out on the droplets. This condensation releases latent heat, and locks the temperature of the vitreous ice droplets to a fixed value. As the expansion continues and the cryogenic gas density drops further, collisions eventually cease and this cryogenic gas coating begins to evaporate. This cools the vitreous ice droplets and continues to do so until the vapor pressure either approaches the ambient pressure or becomes slow on the molecular beam transit time scales. Overall, the cryogenic gas temperature is expected to drop by a factor of 5 to 10 over during the transit through the capillary and the subsequent free-jet expansion. If the vitreous ice droplets are cooled by the same amount, they will reach an end temperature of under ˜500 mK in the fully expanded vitreous ice droplet beam. This end temperature is orders of magnitude lower than is possible with simple evaporative cooling of water droplets in the absence of cryogenic gas.

These methods provide quench-cooling (often called “splat-cooling”) of particles in the droplets, which is desirable for at least the following reasons: (1) Quenching freezes in the native conformation of the particle, such as a protein, protein-ligand complex, or virus; (2) Rapid quenching promotes formation of amorphous or vitreous ice rather than crystalline ice, minimizing the need to add cryoprotectant to the particle solution for this purpose; (3) A low rotational temperature of the particle is crucial to effective laser alignment (for molecular structure determination, as discussed below); and (4) The vapor pressure of the droplet decreases exponentially with temperature, becoming truly UHV-compatible at sufficiently low temperature.

The methods of each of these embodiments of the first aspect can further comprise analyzing or manipulating the particles in the vacuum chamber. Non-limiting examples of particle manipulation or analysis that may require particle injection into vacuum include molecular structure determination, spectroscopy, particle deposition onto a substrate (to produce, for example, sensor arrays), nanoscale free-form fabrication, formation of novel low temperature forms of particle-containing complexes, bombardment of particles by laser light, x-ray radiation, neutrons, or other energetic beams; controlling or promoting directed, free-space chemical reactions, possibly with nanoscale spatial resolution; and separating, analyzing, or purifying these particles.

### Structural Determination

In a preferred embodiment of each of the above embodiments of this first aspect, the method further comprises determining a structure of the particle. Any method for making such a structural determination can be used, including but not limited to methods based on diffraction.

In a preferred embodiment, determining a structure of the particle comprises

(i) passing the vitreous ice particle droplet beam through a laser beam and a diffracting beam to produce a diffraction pattern for a first alignment of a plurality of particles in the vitreous ice particle droplet beam. In a further preferred embodiment, the method further comprises:

(ii) passing the vitreous ice particle droplet beam through the laser beam and the diffracting beam to produce a diffraction pattern for a second alignment of a plurality of particles in the vitreous ice particle droplet beam;

(iii) repeating step (ii) a desired number of times to produce diffraction patterns of further alignments of a plurality of the particles in the vitreous ice particle droplet beam; and

(iv) determining a structure of the particle from a plurality of the diffraction patterns obtained from different alignments of the particle.

In these embodiments, the present invention provides novel methods to promote non-crystallization-based molecular structure determination involving “serial diffraction.” Instead of arranging the molecule, such as a protein, in a three-dimensional periodic array (a crystal), the present methods comprise passing the vitreous ice particle droplet beam comprising many copies of the particle at low temperatures across a high energy diffracting beam, such as a high energy electron or X-ray beam, which produces a scattering (diffraction) pattern. The particles of the vitreous ice particle droplet beam are aligned by a laser beam so that each particle is substantially identically aligned. As used herein, “substantially identically aligned” means that the degree of alignment is sufficient to provide a useful reconstructed image of the average molecular structure from the diffraction patterns. Since there may be many aligned particles in the diffracting beam at any instant, one obtains an average structure from all of these, and the exposure time is reduced by this number. Due to the radiation sensitivity of particles (such as proteins) to electron or X-ray beams, it is not possible to obtain a diffraction pattern from a single particle without destroying it. Thus, the cumulative effect of scattering from many particles (continually being replenished by the vitreous ice particle droplet beam), each subjected to a sub-critical dose of the diffracting beam as it passes through is used. (The “critical dose” is the radiation dose which destroys features of a given size.) In the preferred embodiment, the method comprises collecting diffraction patterns for a plurality of particle alignments by changing the orientation of the laser beam's polarization. These methods provide substantial improvement over previous methods for particle structure determination.

The methods of this structural determination embodiment are conducted at cryogenic temperatures of 200K or below. The particles must be cold, in order to facilitate alignment. The specific temperature used depends on the properties of the particles being analyzed (size, anisotropy, polarizability) and on the power of the focused laser alignment beam, and ranges from near room temperature for viruses such as tobacco mosaic virus (TMV) down to only a few degrees K for small proteins such as lysozyme.

In a further preferred embodiment of structural determination embodiments, a large number of particles (up to many millions per second) are passed through a small volume in space (the “alignment volume”) defined by the focused alignment laser beam. The larger the flux of particles through this alignment volume, the higher the total diffracted intensity and the shorter the measurement time. In a preferred embodiment, the alignment volume is between 5-30 micrometers on a side (more preferably between 5-15 micrometers on a side), and the transverse width of the vitreous ice particle droplet beam is no larger than this.

The structural determination methods comprise passing the vitreous ice particle droplet beam through a laser beam and a diffracting beam. There is no time dependence, and thus any process that provides laser beam-aligned particles in a diffracting beam can be used. One embodiment comprises simultaneous intersection of all three beams in an “intersecting volume,” which includes the alignment volume discussed above. In another embodiment, laser alignment of the particles occurs first, followed by passing the aligned particles through the diffracting beam. In this embodiment, passing the vitreous ice particle droplet beam through a laser beam and a diffracting beam comprises:

(a) passing the vitreous ice particle droplet beam through a laser beam to produce a first alignment of the particles in the vitreous ice particle droplet beam, wherein the first alignment comprises a plurality of the particles with a first alignment;

(b) passing the first alignment of the particles through a diffracting beam to produce a diffraction pattern of the first alignment of the particles;

(c) passing the vitreous ice particle droplet beam through the laser beam to produce second alignment of the particles in the vitreous ice particle droplet beam, wherein the second alignment comprises a plurality of the particles with a second alignment;

(d) passing the second alignment of the particles through the diffracting beam to produce a diffraction pattern of the second alignment of the particle; and

(e) repeating steps (c-d) a desired number of times to produce diffraction patterns of further alignments of a plurality of the particles in the vitreous ice particle droplet beam.

The methods of the structural determination embodiment comprise passing vitreous ice particle droplet beams through a laser beam. Any laser source can be used, so long as the resonant absorption of laser beam energy by ice-jacketed particles is avoided. Optical alignment of particle beams is described in H. Stapelfeldt, Review of Modern Physics, Vol. 75, p. 543, 2003, incorporated herein by reference. In a preferred embodiment, an infrared laser beam is used. In a more preferred embodiment, a continuous (CW) laser source, such as a fiber laser, is used, operating at a wavelength of about one micron, where resonant absorption of laser-beam energy by the ice-jacketed particles is avoided. The laser intensity and temperature needed to achieve a given degree of alignment depend at least on the size of the particle, its shape, and the resolution required in the final reconstructed image of the particle. Useful results can be obtained, for example, with CW infrared lasers whose power is greater than fifty watts. However, it will be understood that other wavelengths of laser light can also be used, where the wavelength of the laser light is chosen such that resonant absorption of laser beam energy by ice-jacketed particles is avoided. Alignment power may be wavelength-dependent. In a preferred embodiment, the upper limit on laser power density is approximately 5×10¹⁴ W/cm². (Larsen, PRL 85 (12) 2000 p. 2470)

It will also be understood that lasers other than fiber lasers can be used. Examples of lasers include, but are not limited to, semiconductor lasers, solid state lasers such as flashlamp pumped lasers or laser-diode-pumped solid state lasers (such as Nd:YAG or the like), and gas lasers. It will also be understood that arrays of lasers can also be used.

It will also be understood that pulsed lasers can also be used. Operation of pulsed lasers and pulse rates are known in the art and can have different pulse lengths and pulse repetition frequencies. When using pulsed lasers, it is preferred that the diffraction patterns are obtained only from time periods wherein the laser pulse is “on” and particles in the vitreous ice particle droplet beam are thus aligned.

The methods of the invention can utilize any laser source as described above, including those placed within the vacuum chamber, or external to the vacuum chamber. Laser beams from such external laser sources can be introduced into the vacuum chamber by any suitable means known in the art, including but not limited to, a fiber-optic feedthrough, a light pipe feedthrough, and a window port of a vacuum chamber that is transparent to the wavelength of the laser light. Additional optics (which could be inside the vacuum or outside the vacuum), such as lenses, may also be used with the laser to control where the beam interacts with the molecules.

It is known in the art that intense laser fields can align molecules along a given space-fixed axis, force them to a plane, or eliminate their rotations altogether, by choice of the polarization to linear, circular, or elliptical, respectively.

A strong linearly polarized laser pulse of sufficiently long duration can align the largest polarizable axis of a particle along a given direction fixed in space. It is also possible to achieve three-dimensional alignment of the direction (but not the sense) of all axes of a polyatomic particle by using an elliptically polarized laser field. The 3D alignment is generally applicable to particles that possess different polarizabilities along three particle axes, which in turn depends on the particle shape for a homogeneous dielectric. The ellipticity necessary to optimize the degree of alignment depends on the specific polarizability tensor and particle shape. “Sense” means the “direction” along a given line—eg north or south on a road which runs in a north-south direction. The ambiguity of sense means, for example, that laser-alignment using linear polarization creates equal populations of particles which are erect and those which are upside down—these have the same energy in the laser field. This leads, in three-dimensions, to six possible orientations for particles aligned with elliptically polarized light. This ambiguity does not prevent the diffraction data from being used to provide a correct three-dimensional map of the particle structure.

As used herein, “aligned” means a defined order of the particle geometry with respect to a space-fixed axis. The set of particles within the diffracting beam are aligned to within an accuracy of a few degrees or better. This accuracy depends on the laser power, the temperature and the size and shape of the particles, as discussed above. The resolution of the final three-dimensional reconstructed image of the particle is approximately equal to this angular spread multiplied by half the length of the particle, and may be less than one nanometer. As the spread is increased (due, for example, to an increase in temperature), the resolution of the final image of the particle becomes degraded. In general, for a given particle, the degree of (adiabatic) alignment can be optimized by increasing the intensity of the alignment field or by lowering the rotational temperature of the particle.

In one non-limiting example, a large particle (such as a ribosome) with an assumed dielectric constant of K=4 passing through a 100 W CW laser focus of 10 micron diameter is cooled to approximately 80K to achieve an alignment error corresponding to a resolution of 0.7 nm. In a further non-limiting example, a small particle (such as lysozyme) under the same conditions is cooled to approximately 10K to achieve the same alignment error.

In another exemplary embodiment, a vitreous ice particle droplet beam velocity of 50 m/sec or less is used, in combination with a CW laser power of 100 W at one micron wavelength, and an interaction volume for the three beams of ten microns on a side.

Based on the teachings herein, those of skill in the art can optimize conditions for structure determination of any particle of interest.

As will be understood by those of skill in the art, a “second alignment” or “further alignments” of the particles are alignments that differ from the first alignment, by changing the orientation of the laser beam's polarization.

In a further preferred embodiment of the structure determination embodiment, passing the vitreous ice particle droplet beam through a laser beam within the vacuum chamber to produce a plurality of aligned vitreous ice particle droplet beams comprises rotating the laser beam in steps about the vitreous ice particle droplet beam to repeatedly change the alignment of the particles in the vitreous ice particle droplet beam. Any number of steps can be used. In various embodiments, the method comprises rotating the laser beam to obtain 2, 3, 6, 18, 45, 90, or 180 alignments of the particles, or as many as are needed for the desired resolution, as understood by those skilled in the art of tomography.

The methods of the structure determination embodiment comprise passing aligned particles in the vitreous ice particle droplet beams through a diffracting beam to produce a diffraction pattern of the aligned particles. The diffracting beam can be any coherent diffracting beam, such as a high energy electron or X-ray beam, wherein “high energy” means energy high enough so that the wavelength is smaller than the particle so that it will diffract. The diffracting beam can comprise continuous or pulsed diffracting beams. For example, a synchrotron can be used to provide a source of pulsed X-ray beams. Pulsing the X-ray beam with many particles in the intersection volume at once can be used, for example, to study the time dependence of electronic processes in a particle, with the great advantage of increased signal due to many particles diffracting at once. So long as a pulse is generated to analyze at least two of the particles in the intersecting volume, a pulsed diffracting beam can be used, preferably in combination with synchronization.

In one preferred embodiment, the diffracting beam comprises an electron beam generated from an electron source. In a further preferred embodiment of this first aspect, a magnetic lens is used to focus the electron beam generated at the electron source. In a still further preferred embodiment of this first aspect, beam deflectors direct the electron beam to intersect the aligned hydrated molecule beam at a desired site.

As noted above, due to the radiation sensitivity of particles, such as proteins, to electron or X-ray beams, it is not possible to obtain a diffraction pattern from a single particle without destroying it, and thus the methods comprise determining the cumulative effect of scattering of many particles, each subjected to a sub-critical dose of the diffracting beam. Such a “sub-critical dose” depends on at least the particle being analyzed and the desired resolution. For example, the resolution needed to see the fold of a protein is approximately 0.7 nm. Determination of the sub-critical dose is well known to those of skill in the art.

The resulting diffraction patterns can be acquired using any appropriate detection system, including but not limited to existing X-ray and electron-area detector systems, which are well known in the art. In a further preferred embodiment, determining a structure of the particle from the plurality of diffraction patterns obtained from different alignments of the particle comprises combining the plurality of diffraction patterns to form a three-dimensional pattern of the particle. The structures can be solved, for example, using these electron diffraction patterns and new iterative solutions to the phase problem (described in S. Marchesini et al., Phys. Rev. B68, P. 140101R (2003) incorporated herein by reference), together with the heavy-atom method. Charge-density maps can be obtained for many orientations of the particle (controlled by the laser polarization), and synthesized tomographically to generate a three-dimensional view.

Imaging particles, such as proteins, in this fashion is an important step in drug design. Images of the protein with and without a docked drug molecule at one of its receptors can help determine what drugs are candidates for protectively blocking receptors susceptible to virus reception.

The structure determination methods of this first aspect do not require crystal formation, are not restricted to proteins that form two-dimensional crystals, and they involves much simpler and more rapid data acquisition than electron cryomicroscopic imaging of macromolecules.

In a second aspect, the present invention provides devices or rapidly forming vitreous ice particle droplets, comprising

(a) a capillary tube comprising a distal end and a proximal end, wherein the distal end is adapted to be in fluid connection with a reservoir adapted to contain a hydrated particle solution, and wherein the proximal end comprises a nozzle;

(b) a first chamber in fluid connection with the nozzle;

(c) a cryogenic gas source, in fluid connection with the first chamber;

(d) an injection tube comprising an inlet and an outlet, wherein the inlet is in fluid communication with the first chamber, and the outlet is adapted to be in fluid communication with a second chamber; and

(d) a housing.

As used herein, the term “in fluid communication with” means a connection that permits the passage of liquids, gases, electrons, or photons between the recited components.

Embodiments of the capillary tube, nozzle, first chamber, and second chamber are described in detail above.

The “reservoir adapted to contain a hydrated particle solution” can be any container that can be fluidly connected to the capillary tube and contain a hydrated solution of the particles.

Similarly, the “cryogenic gas source” gas source can be any container that can dispense the cryogenic gas for use in the methods of the invention, and that can be placed in fluid communication with the first chamber.

The “injection tube” comprises a connection between the first chamber and the second chamber, and can be any type of device that can serve to inject a vitreous ice particle droplet beam from a higher pressure chamber to a lower pressure chamber, such as a vacuum chamber. In a further embodiment of this second aspect, the injection tube has an I.D. of about 100 nm-100 μm.

In a preferred embodiment, the device further comprises a cooling system adapted to keep the first chamber at a temperature of 200K or lower. The device comprises a temperature control system for maintaining a temperature in the vacuum chamber of 200K or less. There are no limitations on the type of system employed, so long as it can keep the vacuum chamber at a temperature of 200K or less. As taught by Faubel, [M. Faubel, et al., Z. Phys. D 10, 269-277 (1988)], water droplets cool extremely rapidly in vacuum, reaching temperatures as low as 200 K, which would give access to the upper end of this range. To reach lower temperatures it is preferred to pass the jacketed proteins through a cryogenic gas to induce additional cooling, as is well known in the art.

In a further preferred embodiment of this second aspect, the device further comprises a second chamber in fluid communication with the outlet of the injection tube; preferably the second chamber is a vacuum chamber. Embodiments of the second chamber are described above and below.

In a further embodiment, the device further comprises a means to perturb the nozzle, including but not limited to a concentric piezoelectric element, allowing small oscillations to be applied to the nozzle tube, triggering the Rayleigh instability at a desired frequency and thereby locking the droplet production to a controllable phase. In a further embodiment, the means is one that can provide an intense, non-periodic excitation to the nozzle to produce droplets on demand. For example, single intense pulses can be used to deliver “droplets on demand” as in ink-jet printers.

In a further embodiment, the device further comprises a means to apply an electric potential to the nozzle. Such means include, but are not limited, to inductive charging devices controlled by a feed-back loop that measures current flow in the vitreous ice particle droplet beam.

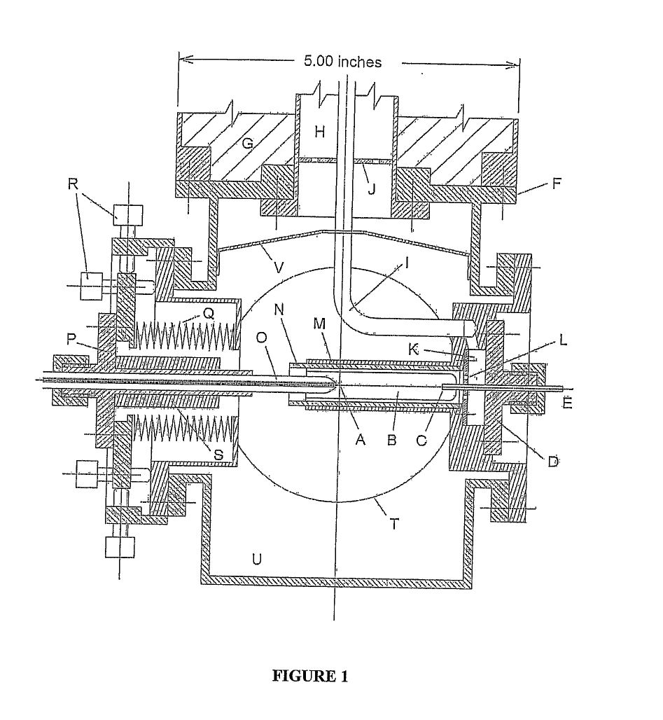

A non-limiting example of the device of the second aspect of the invention is presented in FIG. 1. The hydrated particle solution emerges from a capillary tube (A), in fluid communication with a hydrated particle reservoir (not shown), that has been “pulled” to form a convergent nozzle. Suitable nozzles range in aperture diameters from 100 nm to 100 μm. The hydrated particle solution, preferably at near room temperature, and possibly with cryoprotectant added to the solution of avoid crystallization, passes out of this nozzle and into the first chamber, in this case a partially enclosed gas tube volume (B), through which cold cryogenic gas flows in the opposite direction (right to left in the figure). The pressure and flow velocity of the cryogenic gas can be set independently over a large range, as discussed above. The hydrated particle emerging from the nozzle breaks up due to Rayleigh instability and forms a hydrated particle stream moving along the axis of the helium gas tube (left to right in the figure).

As these particle droplets travel through the cold, dry helium, they cool rapidly by transferring thermal energy to the counter-flowing cold helium gas and by evaporation of water molecules from the droplets. The droplet stream passes into the inlet (C) of the injection tube, a ca. 100 μm I.D. capillary tube. The mount (D) of this tube is designed to allow easy access to and change of the capillary tube. At the far end of the capillary tube (E), the droplet beam emerges into a vacuum of 10⁻⁷ to 10⁻⁶ torr. Capillaries that are slightly convergent on this exit end (E) may be used, to localize the pressure drop at the outlet of the capillary and thereby to maximize expansion cooling. Likewise, the inlet end (C) may be chosen to have a slight outward taper. Further downstream, the droplet jet passes through the wall of a liquid nitrogen shroud (not shown in the figure) and then though a conventional molecular beam “skimmer” and on into the vacuum chamber.

All of the above components are situated within a custom-fabricated housing, the upper flange of which (F) bolts on to the cryogenic gas reservoir (G), for example, a commercial helium cryostat. A hollow tube (H) passes up through the center of the helium reservoir and out to the atmosphere. Passing down the center of this hollow tube is a smaller diameter tube (I), through which cryogenic gas is flowed at a desired pressure. This smaller tube is mounted on the axis of the larger tube by baffles (J) containing staggered apertures to lead to turbulent flow of helium in the outer tube, which in turn leads to good heat transfer from the 4K walls of this tube to the inner tube. The intent is to efficiently cool the helium flowing down the inner tube to 4K, yet not to liquefy this gas. The inner tube feeds the helium gas into a small annular plenum (K) that surrounds the injection tube. This helium gas then passes though apertures in a plate (L) to form the counter-flowing gas for drying and cooling the initial particle droplet stream. The aperture plate can be easily removed and replaced with one of a different hole pattern and hole size. By choice of aperture plate, the pressure and flow rate in the cryogenic gas counter flow can be adjusted independently.

The first chamber (in this case, a helium gas tube) (B) comprises an inner cylindrical sleeve of fused silica (N). This tube is press-fitted into an outer metallic sleeve (M), which has open windows cut along its front and back sides to allow an unobstructed view through the droplet forming region between the nozzle (A) and the entrance to the injection tube (C). Via windows on the front and back sides of the cryohousing (T) and corresponding windows through the liquid nitrogen shroud, it is therefore possible to observe the droplet beam by use of a commercial microscope of appropriately large working distance. The beam is backlighted from behind for this purpose (from the far side of the assembly in FIG. 1).

Gas passing out of the helium flow tube flows up the exit tube (H). A small dome (V) is press-fitted into the upper portion of the cryohousing to avoid having helium condensate from the 4 K walls drip onto nozzle and drying/cooling tube. Any such condensate is thereby ducted to the walls and through appropriate holes down to the bottom of the cryohousing (U), where ample reservoir capacity has been provided to accommodate an appreciable volume of liquid helium. Ideally, there will be will little or no condensate.

The nozzle tube is optionally surrounded by coaxial heater (O) to prevent the particle solution from freezing in the nozzle. Insulation, possibly vacuum insulation, is provided to minimize heat flow into the cryohousing, as this would increase liquid helium consumption. This heating and insulation around the liquid supply line must continue through the wall of the cryohousing and out through the liquid nitrogen-cooled radiation shield (not shown in the figure) that surrounds the cryohousing.

A concentric piezoelectric element (S) allows small oscillations to be applied to the nozzle tube, triggering the Rayleigh instability at a desired frequency and thereby locking the droplet production to a controllable phase. This is similar to the mechanism employed by Trostell [7]. Stroboscopic and other phase lock-in techniques can then be utilized in the beam analysis or use.

The nozzle assembly (P) mounts into the cryohousing by means of a bellows (Q). The nozzle assembly is supported on a mechanism (R) that allows limited translation along the axis of the beam and also limited motion in two orthogonal lateral directions. The push/pull jack screws also allow limited tilt adjustment of the axis of the nozzle. Provided the liquid beam emerges reasonably parallel to the axis of the tube, this motion should allow the beam to be directed down the axis of the injection tube while observing the beam through the windows in the cryohousing.

In a third aspect, the present invention provides devices for carrying our serial diffraction, comprising:

(a) a vacuum chamber;

(b) a diffracting beam source in fluid communication with the vacuum chamber,

(c) a laser beam source in fluid communication with the vacuum chamber;

(d) a vitreous ice particle droplet beam source, comprising the device disclosed in the second aspect of the invention, wherein the outlet of the injection tube is in fluid communication with the vacuum chamber;

(e) a temperature control system for maintaining a temperature in the vacuum chamber of 200K or less; and

(f) a detector system in connection with the vacuum chamber;

wherein the diffracting beam source, the laser beam source, and the vitreous ice particle droplet beam source are positioned to permit beams directed from the diffracting beam source, the laser beam source, and the vitreous ice particle droplet beam source to intersect in the vacuum chamber in an intersecting volume of between 10 μl and 100 μL; and wherein the detector system is positioned so as to receive diffraction patterns from molecules in the molecule beam passing through the diffracting beam.

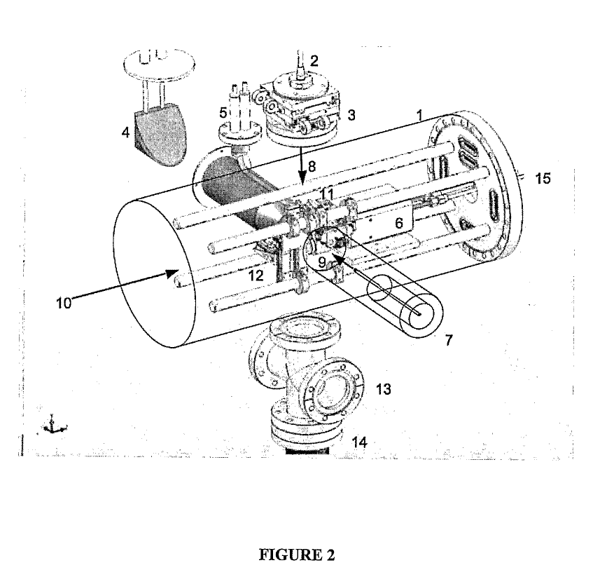

A non-limiting example of a device for carrying out the methods of the invention is provided in FIG. 2. The outline of the vacuum chamber (1) is shown. As is well known in the art, a vacuum chamber is a device to create a partial volume of space that is empty of matter and radiation, including air, so that gaseous pressure is much less than standard atmospheric pressure. Any such vacuum chamber can be used in conjunction with the methods of the invention, so long as a temperature of 200K or below in the vacuum chamber can be maintained.

A laser source is provided that is in fluid communication with the vacuum chamber. As used herein, the term “in fluid communication with” means a connection that permits the passage of liquids, gases, electrons, or photons between the recited components. The laser source may comprise a docking port for attachment of an external laser source, may comprise the docking port and the external laser source, or may comprise a laser source within (either partially or completely) the vacuum chamber. FIG. 1 shows an exemplary external laser source (2) is shown in fluid communication with the vacuum chamber. In the example, the laser source (2) is an infrared continuous fiber laser of 100 W and 1 micron wavelength. Laser beams from such external laser sources can be introduced into the vacuum chamber by any suitable means known in the art, including but not limited to, a fiber-optic feedthrough, a light pipe feedthrough, and a window port of a vacuum chamber that is transparent to the wavelength of the laser light. Additional optics (which could be inside the vacuum or outside the vacuum), such as lenses, may also be used with the laser to control where the beam interacts with the molecules. In the example provided in FIG. 1, the external laser source (2) comprises a positioning device, laser window and quarter wave plate (3) for producing elliptically polarized laser fields.

A laser beam (8) is produced from the laser source and passes into the vacuum chamber (1). In this example, directly opposite the entry point of the laser beam into the vacuum chamber is a laser dump (14), in this case a water-cooled infra-red laser beam dump, to capture the beam after passage through the intersecting volume. In this example, the laser beam dump (14) is internal to the vacuum chamber but water-cooled from the outside, with heat transferred across a vacuum seal. While preferably placed directly opposite the laser source, the laser beam dump can be placed at any position from which it can collect the laser beam after passage through the intersecting volume. The laser dump can further comprise an observation port.

As noted above in the first aspect of the invention, any laser source can be used, so long as the resonant absorption of laser beam energy by ice-jacketed particles is avoided. In a preferred embodiment, an infrared laser beam is used. In a more preferred embodiment, a continuous (CW) laser source, such as a fiber laser, is used. It will also be understood that lasers other than fiber lasers can be used. Examples of lasers include, but are not limited to, semiconductor lasers, solid state lasers such as flashlamp pumped lasers or laser-diode-pumped solid state lasers (such as Nd:YAG or the like), and gas lasers. It will also be understood that arrays of lasers can also be used. It will also be understood that pulsed lasers can be used.

It is highly preferred that the laser beam polarization can be rotated in steps about the beam of particles to repeatedly change the alignment of the molecules so that diffraction patterns are accumulated from many aligned particles at each new orientation. In a preferred exemplary embodiment the laser beam polarization can be rotated in 1° increments throughout 180° using a quarter-wave plate, as is well known to those skilled in the art of laser optics, to form 180 different diffraction patterns. The quarter-wave plate, which controls polarization of the laser, is situated near the laser beam positioning device (3) in FIG. 1. These different diffraction patterns are subsequently combined to form a three dimension map of the diffraction pattern of the molecule. Any means by which such rotation of the laser beam polarization can be carried out can be used with the present invention. In another embodiment, the laser beam source itself can be rotated relative to the intersecting volume.

The laser beam-dump (14) device may also comprise an observation port (13) in FIG. 2 for a visible alignment laser (13). For example, a visible laser co-aligned with the infrared laser can be used to align the laser optics to the droplet beam. This beam (with the powerful infrared laser switched off) can safely be observed by eye through the window at 13, which views an inclined mirror, directing the view up into the visible laser coming down from 8 when a droplet beam crosses this visible laser beam, it produces a characteristic scattering pattern on a semi-transparent viewing screen inserted horizontally in the upper neck of the “cross” 13. By adjusting the lateral position and focus of the laser beam for the strongest such scattering pattern (known in the art of optics as a “Fraunhoffer diffraction pattern”), the visible laser (and therefore the co-aligned infra-red laser) can be aligned with the droplet beam.

The vitreous ice particle droplet beam source may comprise may comprise a docking port and an external vitreous ice particle droplet beam source as disclosed in the second aspect of the invention above, or may comprise the vitreous ice particle droplet beam source within (either partially or completely) the vacuum chamber. In the example shown in FIG. 1, the vitreous ice particle droplet beam source comprises the device of the second aspect of the invention, wherein the outlet of the injection tube of the vitreous ice particle droplet beam source is in fluid communication with the vacuum chamber (7). The vitreous ice particle droplet beam source produces a vitreous ice particle beam (9) as described in the first and second aspects of the invention.

FIG. 1 also shows the inclusion of a liquid nitrogen cooled vitreous ice particle droplet beam dump (4) to collect vitreous ice particle droplets after passing through the intersecting volume. This dump also serves to maintain the desired vacuum level in the vacuum chamber. While preferably placed directly opposite the vitreous ice particle droplet beam source, the vitreous ice particle droplet beam dump can be placed at any position from which it can collect the vitreous ice particle droplet beam after passage through the intersecting volume.

As the vitreous ice particle droplet beam passes through the vacuum chamber, evaporation occurs. Thus, the device may further comprise a cryoshield (not numbered; directly below (5)) to prevent buildup of condensation, which can damage, for example, portions of the detector system (6) and (15). Such a cryoshield preferably comprises a cooling source, such as a liquid nitrogen source (5).

In the example shown in FIG. 2, the diffracting beam source (10) is external to and in fluid communication with the vacuum chamber. The diffracting beam source may comprise a docking port for attachment of an external diffracting beam source, may comprise the docking port and the external diffracting beam source, or may comprise a diffracting beam source within (either partially or completely) the vacuum chamber. The diffracting beam source can be comprise any coherent diffracting beam source, such as a high energy electron or X-ray beam, wherein “high energy” means an energy high enough so that the wavelength is smaller than the particle so that it will diffract. The diffracting beam can comprise continuous or pulsed diffracting beams. For example, a synchrotron can be used to provide a source of pulsed X-ray beams. Pulsing the X-ray beam with many particles in the intersection volume at once can be used, for example, to study the time dependence of electronic processes in a particle, with the great advantage of increased signal due to many particles diffracting at once. So long as a pulse is generated to analyze at least two of the particles in the intersecting volume, a pulsed diffracting beam can be used, preferably in combination with synchronization.

In one preferred embodiment, the diffracting beam comprises an electron beam generated from an electron source. In a further preferred embodiment of this first aspect, a magnetic lens is used to focus the electron beam generated at the electron source. In a still further preferred embodiment of this first aspect, beam deflectors direct the electron beam to intersect the aligned vitreous ice particle droplet beam at a desired site, and the size of the diffraction (scattering) pattern formed by the electron is magnified by magnetic lenses, well known to those skilled in the art of electron optics.

The device further comprises a detector system in connection with the vacuum chamber. Any type of detection system capable of detecting and recording molecule diffraction patterns can be used, including but not limited to existing X-ray and electron area-detector systems, which are well known in the art. An exemplary detector system is shown in FIG. 2, comprising a phosphor screen (or similar) (15) coupled by fiber optics to an area detector such as a charge coupled device (6). A computer connected to the detector can then be used to assemble the many two-dimensional diffraction patterns into one three-dimensional pattern. This three-dimensional pattern is then converted into a three-dimensional map of charge-density showing the atomic arrangement using the iterative algorithm of Marchesini and others in Phys. Rev. B68, PAGE 140101 (2003), and its three-dimensional extensions (taught, for example, by Millane (J. Opt. Soc. Am. A13, P. 725 (1996), incorporated herein by reference and attached) to solve the phase problem for the continuous distribution of scattering from an isolated object. Each diffraction pattern formed at each step is formed by numerous particles diffracting the diffracting beam, such as a collimated electron beam, so that an image of sufficient detail (resolution) and contrast can be reconstructed after passing these diffraction patterns through an iterative phasing algorithm (described in Marchesini et al.). Examples of electron diffraction patterns from partially aligned particles in a particle beam are to be found in K. Hoshina et al., J. Chem. Phys., 118, p. 6211 (2003), incorporated by reference and attached. Magnetic lenses can be used to focus the electron beam generated at an electron source. Beam deflectors can direct the beam to intersect the particle beam.

The device comprises a temperature control system for maintaining a temperature in the vacuum chamber of 200K or less. There are no limitations on the type of system employed, so long as it can keep the vacuum chamber at a temperature of 200K or less. As taught by Faubel, [M. Faubel, et al., Z. Phys. D 10, 269-277 (1988)], water droplets cool extremely rapidly in vacuum, reaching temperatures as low as 200 K, which would give access to the upper end of this range. To reach lower temperatures it is preferred to pass the jacketed proteins through a cryogenic gas to induce additional cooling, as is well known in the art.

The device is configured so that beams directed from the diffracting beam source, the laser beam source, and the vitreous ice particle beam source intersect in the vacuum chamber in an intersecting volume of between 10 μL and 100 μL; and wherein the detector system is positioned so as to receive diffraction patterns from particles in the particle beam passing through the diffracting beam. In a preferred embodiment, the device further comprises positioning means located with the vacuum chamber, for precise positioning of the beams. In one embodiment, the positioning means comprises electrical motors. One such electrical motor is used to focus the laser beam at the same height as the vitreous ice particle beam. (See FIG. 2: the lateral position of the laser beam is adjusted by motors (3) which move it sideways). Other motors inside the chamber are positioned to move the vitreous ice particle beam as needed, for example in two orthogonal directions. In a further embodiment, the entire vacuum chamber itself may be moved by external positioning screws to provide needed beam positioning, such as centering the diffracting beam along the axis of the vacuum chamber. It is further preferred that the positioning means is controlled by signals from a computer connected to the vacuum chamber by electrical feedthroughs, as is well known to those skilled in the art of vacuum electronics.

In a further preferred embodiment, the device further comprises a beam-stop inside the vacuum chamber, which can also be moved by internal motors. The beam stop can be positioned, for example, in the center of the area detector (6) to prevent it being overloaded by the unscattered portion of the diffracting beam.

Any embodiments of diffracting beam sources, laser beam sources, vitreous ice particle beam sources, vacuum chambers, temperature control means for producing a temperature in the vacuum chamber of 200K or less, and detector systems described above in the first and/or second aspect of the invention can be used with the device of the third aspect of the invention.

We claim:

1. A method for rapidly forming a vitreous ice particle droplet beam, comprising contacting a hydrated particle droplet beam with a cryogenic gas in a first chamber at a temperature of 200K or lower, and at a pressure of 1 atm or lower, thereby forming a vitreous ice particle droplet beam.

2.-7. (canceled)

8. The method of claim 1, further comprising injecting the vitreous ice particle droplet beam into a second chamber, wherein the second chamber has a temperature of 200K or lower, and wherein the second chamber has a pressure suitable to maintain the vitreous ice particle droplet beam.

9. The method of claim 8, wherein the second chamber comprises a vacuum chamber.

10. The method of claim 8 wherein the injecting comprises passing the vitreous ice particle droplet beam through a capillary with an inner diameter of 100 μM or less, and injecting the vitreous ice particle droplet beam from the capillary into the second chamber.

11. The method of claim 9, wherein the pressure in the second chamber is between 10⁻⁵ torr and 10⁻⁸ torr.

12.-13. (canceled)

14. The method of claim 1, further comprising determining a structure of the particle.

15. The method of claim 14, wherein determining a structure of the particle comprises
(i) passing the vitreous ice particle droplet beam through a laser beam and a diffracting beam to produce a diffraction pattern for a first alignment of a plurality of particles in the vitreous ice particle droplet beam.

16. The method of claim 15, further comprising:
(ii) passing the vitreous ice particle droplet beam through the laser beam and the diffracting beam to produce a diffraction pattern for a second alignment of a plurality of particles in the vitreous ice particle droplet beam;
(iii) repeating step (ii) a desired number of times to produce diffraction patterns of further alignments of a plurality of the particles in the vitreous ice particle droplet beam; and
(iv) determining a structure of the particle from a plurality of the diffraction patterns obtained from different alignments of the particle.

17. The method of claim 14 wherein each vitreous ice particle droplet has a diameter of 1 μm or less.

18. The method of claim 15, wherein passing the vitreous ice particle droplet beam through a laser beam and a diffracting beam comprises simultaneous intersection of all three beams in an intersecting volume.

19. The method of claim 15, wherein passing the vitreous ice particle droplet beam through a laser beam and a diffracting beam comprises:
(a) passing the vitreous ice particle droplet beam through a laser beam to produce a first alignment of the particles in the vitreous ice particle droplet beam, wherein the first alignment comprises a plurality of the particles with a first alignment;
(b) passing the first alignment of the particles through a diffracting beam to produce a diffraction pattern of the first alignment of the particles;
(c) passing the vitreous ice particle droplet beam through the laser beam to produce second alignment of the particles in the vitreous ice particle droplet beam, wherein the second alignment comprises a plurality of the particles with a second alignment;
(d) passing the second alignment of the particles through the diffracting beam to produce a diffraction pattern of the second alignment of the particle; and
(e) repeating steps (c-d) a desired number of times to produce diffraction patterns of further alignments of a plurality of the particles in the vitreous ice particle droplet beam.

20.-22. (canceled)

23. The method of claim 15, wherein the method comprises producing at least 6 diffraction patterns of different alignments of a plurality of the particles in the vitreous ice particle droplet beam.

24. The method of claim 15, wherein the diffracting beam comprises an electron beam.

25. The method of claim 15, wherein the diffracting beam comprises an X-ray beam.

26. The method of claim 15, wherein determining a structure of the molecule comprises combining the plurality of diffraction patterns to form a three-dimensional pattern of the particle.

27.-28. (canceled)

29. The method of claim 1, wherein the particle comprises a protein.

30. The method of claim 1, wherein the particle comprises a virus.

31. A device for rapidly forming vitreous ice particle droplets, comprising
(a) a capillary tube comprising a distal end and a proximal end, wherein the distal end is adapted to be in fluid connection with a reservoir adapted to contain a hydrated particle solution, and wherein the proximal end comprises a nozzle;
(b) a first chamber in fluid connection with the nozzle;
(c) a cryogenic gas source, in fluid connection with the first chamber;
(d) an injection tube comprising an inlet and an outlet, wherein the inlet is in fluid communication with the first chamber, and the outlet is adapted to be in fluid communication with a second chamber; and
(d) a housing.

32. The device of claim 31, further comprising a cooling system adapted to keep the first chamber at a temperature of 200K or lower.

33. The device of claim 31, wherein the injection tube has an I.D. of between 100 nm and 100 μm.

34. The device of claim 31, further comprising a second chamber in fluid communication with the outlet of the injection tube.

35. The device of claim 34, wherein the second chamber comprises a vacuum chamber.

36. The device of claim 31, further comprising a means to perturb the nozzle.

37. The device of claim 31, further comprising a means to apply an electric potential to the nozzle.

38. A device for carrying our serial diffraction, comprising:
(a) a vacuum chamber;
(b) a diffracting beam source in fluid communication with the vacuum chamber,
(c) a laser beam source in fluid communication with the vacuum chamber;
(d) a vitreous ice particle droplet beam source, comprising the device of claim 31, wherein the outlet of the injection tube is in fluid communication with the vacuum chamber;
(e) a temperature control system for maintaining a temperature in the vacuum chamber of 200K or less; and
(f) a detector system in connection with the vacuum chamber;
wherein the diffracting beam source, the laser beam source, and the vitreous ice particle droplet beam source are positioned to permit beams directed from the diffracting beam source, the laser beam source, and the vitreous ice particle droplet beam source to intersect in the vacuum chamber in an intersecting volume of between 10 μl and 100 μL;
and wherein the detector system is positioned so as to receive diffraction patterns from molecules in the molecule beam passing through the diffracting beam.
